(19) United States
(12) Patent Application Publication
(10) Pub. No.: US 2020/0040394 A1
(43) Pub. Date: Feb. 6, 2020

(54) Systems and Methods for Unifying Assemblies that Share Common Taggant Identifier Data

(71) Applicant: Invisidex, Inc., Ojai, CA (US)

(72) Inventors: Reep Paul, Ojai, CA (US); Kabir Sagoo, Ojai, CA (US)

(21) Appl. No.: 16/529,483

(22) Filed: Aug. 1, 2019

Related U.S. Application Data
(60) Provisional application No. 62/713,996, filed on Aug. 2, 2018.

(51) Int. Cl. C12Q 1/6876; C09J 11/08; C09J 177/00; G09C 1/00; G06Q 30/00

(52) U.S. Cl. CPC C12Q 1/6876; C09J 11/08; C09J 177/00; C09J 2477/00; G06Q 30/0185; C09J 2489/00; G09C 1/00

(57) ABSTRACT

The present invention describes systems and methods for embedding authentication data into a quality control tamper evidence adhesive sealant material used to mark a system of parts and assemblies, creating a unified and traceable layer of security data to identify unauthorized modifications to manufactured systems.

### 1. FIELD OF THE INVENTION

The present invention relates to the fields of chemistry, biochemistry, material science, analytical tools, microbiology, adhesives, and sealants. The present invention also relates to supply chain management, authentication, anti-counterfeiting, quality control, tamper deterrence, product traceability, and computer networks.

### 2. BACKGROUND

Inspection paints described in prior art are used in many industrial applications to mark items for visual inspection and tamper identification. Inspection paint can be applied to nuts, bolts, critical fasteners, and other coupled parts whereby the inspection paint creates a seal between two adjacent parts. The inspection paint dries and becomes brittle. If upon visual inspection the seal is observed to be broken, then it is reasonably assumed the part has been tampered with or the coupled parts no longer conform to specification such as torque value as a result of vibrational loosening, tampering, sabotage, or some other force.

Inspection paints are routinely used by aircraft, shipping, automotive, manufacturing, and other industries to mark items at the original equipment manufacturer (OEM) production level. Authorized OEM personnel apply a mark of inspection paint after the Quality Control team confirms parts conform to their specification. Further down the supply chain, maintenance and repair facilities use inspection paints to confirm parts were repaired back to specification. In the oil and gas industry, pipeline nuts and bolts may need to be repaired and reinstalled, and then marked by authorized service personnel.

Industry currently uses special tapes or labels to protect packaging or external product compartments. If the seal created by tapes and labels breaks, then the OEM may not guarantee warranty. However, inspection paint may also be applied to assemblies and manufactured components below external product surfaces. Therefore, it is more difficult to know if unauthorized modifications have occurred even if the anti-tamper label or tape is intact. Inspection paints may indicate a nut or bolt is out of torque specification and torque needs to be reapplied if the inspection paint seal is broken and not aligned. Furthermore, inspection paint may be applied over electrical connectors to indicate electrical work has been performed or to warn others not to modify the electrical component.

Industries such as commercial aviation, aerospace and defense, automotive, shipping, and manufacturing have such diverse repair and modification points once a manufactured item leaves the original equipment manufacturer (OEM) level. Aircraft are routinely repaired and inspected at maintenance repair and overhaul (MRO) facilities by authorized personnel. Also, grounded aircraft can be breached by unauthorized personnel that attempt to switch out parts or damage the aircraft.

Furthermore, even if inspection paint is intact, it is impossible to tell if it is original. Inspection paint applied subsequent to the original manufacture may be unauthorized or designed to conceal the introduction of a substandard part.

Since there is no identifier data that indicates which entity applied the inspection paint, OEMs are faced with warranty fraud, fraudulent claims of product integrity or failure, product tampering, and unauthorized repairs.

### SUMMARY OF THE INVENTION

In various embodiments, the present invention relates to analyzing data associated with taggants contained within an adhesive sealant used as an inspection paint or witness mark for authenticating performed work and forensically linking configuration management and control assemblies. In particular the present invention relates to embedding identification data into an adhesive sealant material that is extruded onto various surfaces of assemblies to enable visual inspection of tampering and quality control while creating a forensic layer of authentication data between parts and assemblies within manufactured systems using taggant identifier data within the adhesive sealant.

The present invention allows for the interrogation of taggant data contained within an adhesive inspection sealant applied throughout manufactured assemblies systems and subsystems for traceability and identifying if an original equipment manufacturer (OEM) or unauthorized party performed work on the system.

In an embodiment of the present invention, the adhesive sealant contains embedded nucleic acids such as deoxyribonucleic acid (DNA) or ribonucleic acid (RNA). The DNA taggants are introduced to the adhesive chemistry formulation in a colloidal slurry during the production process to ensure homogeneous disbursement throughout the formulation.

DNA taggants are used in the anti-counterfeiting market because of their scalability and ability to hide in plain site: DNA sequences are concealed within host materials and cannot be decrypted unless an interrogator has the correct test kits with the required analytical chemistry.

DNA taggants are short nucleic acid fragment data sequences that can be associated with a lookup table for product identification data. The data contained in DNA taggants can be read using a polymerase chain reaction (PCR) instrument or sequencing device.

Prior art describes the use of DNA taggants for uniquely identifying a product or batch of products; however, manufactured assemblies whose parts are configured to a configuration management and control scheme require an extra layer of security. Since there is a critical need to identify if unauthorized personnel attempted to replace parts with substandard parts and re-apply inspection paint, the DNA data contained in inspection paint used throughout assemblies unifies all the manufactured parts with a layer of authentication data.

Configuration management and control governs specifications of various parts and spares as well as the conditions which parts are modified, introduced, or released into the manufacturing process. Taggants can be specific to an aircraft or a subassembly. For example, an airplane fuselage can be assigned its own taggant material. Later, critical parts can be marked only with the same material containing an identical taggant that matches the original parts and configuration. Inspection paint that contains taggants such as DNA help avoid contaminating the spare parts associated with a manufacturer's configuration management and control scheme in case parts from other sources are used.

In another embodiment of the present invention, surrogate identification data such as primers and probes associated with DNA sequences can be introduced to the adhesive sealant chemistry formulation during production. This prevents analytical equipment outputting a DNA sequence being used to identify and replicate the sequence.

### DETAILED DESCRIPTION OF THE PREFERRED EMBODIMENTS

In various embodiments, the invention relates to analyzing data associated with taggants contained within an adhesive sealant for authenticating performed work and forensically linking parts and assemblies according to their configuration management and control scheme throughout the lifecycle of the manufactured good. In particular the present invention relates to embedding identification data into an adhesive sealant material that is extruded onto various surfaces of assemblies to enable visual inspection of tampering and quality control while creating a forensic layer of authentication data, unifying parts and assemblies within manufactured systems using taggant identifier data.

The present invention allows for the interrogation of taggant data contained within an adhesive inspection sealant applied throughout manufactured assemblies systems and subsystems for traceability and determining if an original equipment manufacturer (OEM) or unauthorized party performed work on the system.

Molecular markers and taggants described in prior art can be embedded into products and various mediums to embed unique identifier data for one item or a batch or items. The identifier data enables product traceability for anti-counterfeiting purposes.

Various anti-counterfeiting technologies were developed to mitigate the risk of counterfeit products entering supply chains. Packaging technologies such as holograms, barcodes, QR codes, special pigments and dyes protect product packaging, but do not authenticate the physical product. Furthermore, counterfeiters are mimicking packaging technologies meant to authenticate goods, thereby defeating the reliability of package-only protection technologies.

Physical and chemical taggants have been developed to protect at-risk materials such as fuels, bulk chemicals, and other products. These taggants can be composed of organic molecules, inorganic fluorescent markers, or nucleic acids such as deoxyribonucleic acid (DNA). Conventional taggant technologies contain data about products or they are associated with authentication and traceability data. In simple systems, if the taggant is present but does not contain data that can be interrogated and decrypted, its mere presence may indicate a product is authentic.

DNA authentication technologies are gaining traction in various industries because of DNA's scalability as well as its ability to hide in plain sight; DNA can be embedded in materials without disclosing the exact DNA sequence. Like barcodes in the super market or unique fingerprints, DNA sequences are data codes that can be customized for a unique authentication signature.

DNA sequences are synthesized into physical DNA molecules. The synthesized DNA molecules can be embedded into materials such as chemicals or surface witness marks for authentication purposes. Due to the prevalence of DNA in environmental conditions, unless a user knows the target sequence they are looking for, it is nearly impossible to interrogate a DNA taggant using the polymerase chain reaction (PCR) analytical method.

Industrial applications may not rely on authenticating one part or item. Rather, manufactured assemblies and sub-assemblies may be comprised of various parts that adhere to a configuration management and control scheme. For example, aircraft configuration management and control governs specifications of various parts and spares as well as the conditions in which parts are modified, introduced, or released into the manufacturing process. Taggants can be specific to an aircraft or a subassembly. For example, an airplane fuselage can be assigned its own taggant material. Later, critical parts can be marked only with the same material containing an identical taggant that matches the original parts and configuration.

Primers and probes hybridize with complementary nucleotides of the target DNA sequence. In PCR, primers and specifically probes bind to the correct DNA fragment assuming the adenine, thymine, guanine, and cytosine pairs match. Probes are used in the PCR process to detect the target sequence and help with target sequence detection.

Instead of compromising the DNA taggant's authentication security sequence by embedding DNA taggants into a mark of inspection paint, primers and probes would solve possible sequence decryption by obfuscating the DNA taggant's sequence. Primers and probes are designed to be specifically compatible with a unique nucleic acid sequence. In an embodiment of the present invention, an operator would interrogate the primer and probe chemistry present in a given sample, match that data to a computer networked system lookup table, and determine if the target DNA sequence matches the witness mark's unique primer and probe signature.

The present invention covers another embodiment wherein designer molecules are uniquely synthesized to pair with a DNA sequence. For example only, pyrrole-imidazole (Py-lm) polyamides recognize and bind DNA with specific sequence characteristics. They are aromatic ring, synthetic small molecules that have a high affinity of binding with compatible DNA double helixes.

Py-lm molecules can be embedded within various products, fluids, bulk chemicals, and other materials for authentication purposes when associated with a given DNA sequence. Py-lm data can protect the identity of a DNA sequence associated with a certain authentication security scheme.

The compounds used to formulate the present invention aid in the homogenous distribution, suspension, durability, and viability of the taggant. In order to achieve this, a colloid was developed. Colloid solvent chemistry was also selected to help blending and ballasting of taggant particles, and maintain consistent and event distribution during manufacturing, shipping, handling, long term storage, prior to, and after the present invention has been opened and partially used. Tack time under 30 seconds was needed, while at the same time, quick and simple solvent sample extraction that is uniform of the taggant was necessary.

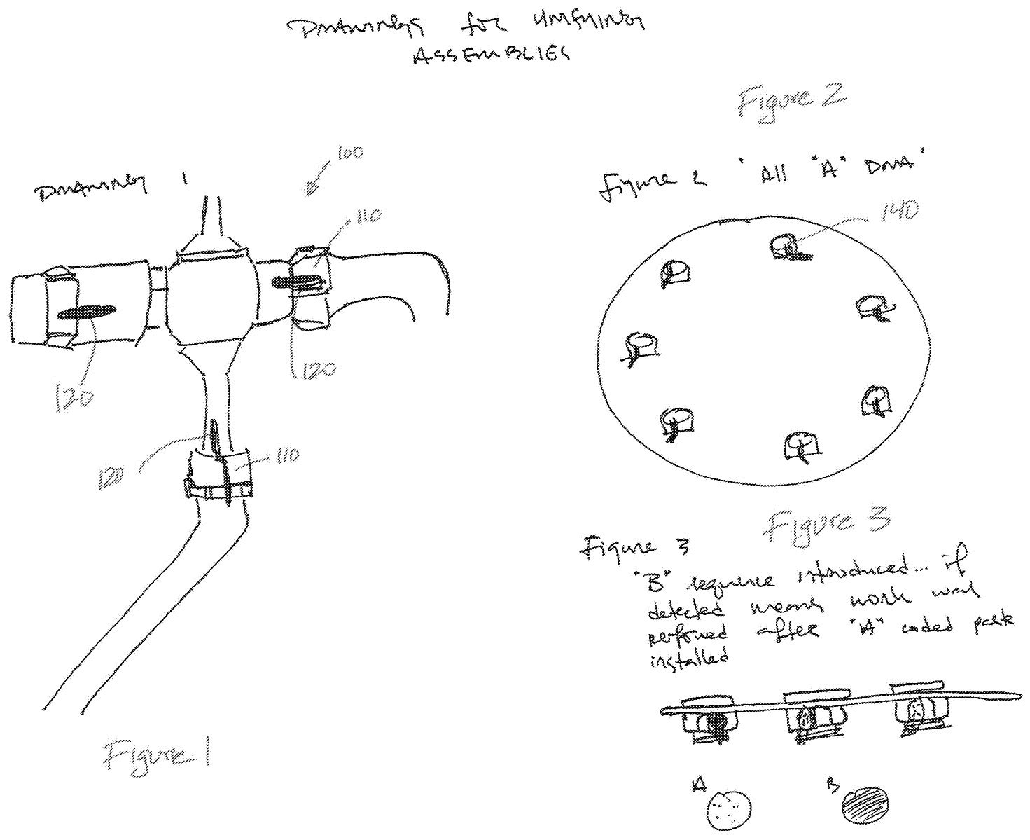

Turning now to FIG. 1, a piping system 100 is shown having torque specific couplings 110. Adhesive sealant 120 having molecular taggants contained therein are shown applied to the surface. These serve as witness marks for tempering, but more importantly, are sophisticated evidence of the authenticity of the parts making up piping system 100. The sealant may be integrated and a DNA sequence verified through PCR.

In FIG. 2, a known DNA sequence “A” is present in the sealant used on all of the nuts 140 after assembly.

In FIG. 3, it has been determined that “B” DNA is present on some bolts. If part of a maintenance plan, the pressure of “B” DNA indicates that authorized work was performed subsequent to the original installation. If no DNA is present, the work was not authorized.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by letters-patent is:

1. An authentication security protection system that unifies parts and assemblies marked with adhesive sealant containing common taggant identification data comprising:
an adhesive sealant applied to parts and assemblies; and
a security taggant additive contained with the sealant.

2. The system of claim 1, wherein the security taggant additive is a nucleic acid.

3. The system of claim 2, wherein the nucleic acid is deoxyribonucleic acid (DNA).

4. The system of claim 3, wherein the security taggant additive DNA information to confirm a system of parts and assemblies are traceable and configured according to a configuration management and control scheme.

5. The system of claim 2, wherein the nucleic acid is ribonucleic acid (RNA).

6. The system of claim 5, wherein the security taggant additive RNA encodes information to confirm a system of parts and assemblies are traceable and configured according to the configuration management and control scheme.

7. The system of claim 1, wherein the security taggant additive is neither DNA nor RNA, but instead is surrogate data associated with the nucleic acid data to obfuscate and protect the integrity of the nucleic acid data signal.

8. The system of claim 7 where the surrogate data are primers and probes compatible with a nucleic acid sequence.

9. The system of claim 7, where the surrogate data are pyrrole-imidazole (Py-lm) polyamides.

10. The system of claim 7, where the surrogate data taggants are introduced to the adhesive sealant chemistry during formulation.

11. The system of claim 7, wherein the surrogate data encodes information to confirm a system of parts and assemblies are traceable and configured according to a configuration management and control scheme.

12. The system of claim 1, wherein the solvent is an ester group.

13. The system of claim 1 where the security taggant additive is blended with a solvent during formulation to promote uniform homogeneity of the taggant's presence in a mark of inspection paint.

14. The system of claim 1, further comprising a synthetic colorant.

15. The system as recited in claim 14, wherein the synthetic colorant yields a transparent look.

16. The system as recited in claim 15, wherein the synthetic colorant is determined by the color code used by a brand's logo so that brand has its own matching adhesive sealant color.

17. The system of claim 1 where the security taggant additive is extracted from the adhesive sealant with a solvent extraction kit.

18. The system of claim 1 where the adhesive sealant with security taggant additives is used to mark a system of parts and assemblies for tamper evidence or vibrational loosening.

19. The system of claim 1, wherein the taggants of adhesive sealant are in a colloidal suspension.

20. A method for formulating an adhesive sealant for an authentication protection system comprising:
creating a colloidal suspension;
adding a molecular taggant;
adding a colorant; and
allowing the formulation to become a paste capable of drying to a tack state in less than 30 seconds.
